(19) United States
(12) Patent Application Publication
(10) Pub. No.: US 2022/0270708 A1
(43) Pub. Date: Aug. 25, 2022

(54) Drug-Food Interaction Prediction

(71) Applicant: Northeastern University, Boston, MA (US)

(72) Inventors: Michael L. Sebek, Boston, MA (US); Albert-László Barábasi, Brookline, MA (US); Giulia Menichetti, Boston, MA (US); Peter Ruppert, Chestnut Hill, MA (US); Italo Faria Do Valle, Boston, MA (US)

(21) Appl. No.: 17/596,791

(22) PCT Filed: Jun. 19, 2020
(86) PCT No.: PCT/US2020/038681

§ 371 Date: Dec. 17, 2021

Related U.S. Application Data
(60) Provisional application No. 62/864,172, filed on Jun. 20, 2019.

(51) Int. Cl. G16B 20/00; G16B 5/20; G16B 45/00

(52) U.S. Cl. CPC G16B 20/00; G16B 5/20; G16B 45/00

(57) ABSTRACT

Methods and systems for filtering data in a protein-protein interaction network are provided, which can be used to identify potential food-drug interactions. A method of filtering data in protein-protein interaction network includes mapping proteins associated with a plurality of chemicals of a first type (e.g drugs) and proteins associated with one or more chemicals of a second type (e.g., foods). The method further includes determining proximities of proteins associated with the plurality of chemicals of the first type and proteins associated with the one or more chemicals of the second type and generating a reduced dataset of proteins within the protein-protein interaction network. The reduced dataset includes proteins associated with a subset of the plurality of chemicals of the first type based on the determined proximities.

### RELATED APPLICATION

This application claims the benefit of U.S. Provisional Application No. 62/864,172, filed on Jun. 20, 2019. The entire teachings of the above application are incorporated herein by reference.

### BACKGROUND

Drugs are widely used for a variety of therapeutic purposes, and it is known that drug interactions can occur with other chemicals, such as chemicals found in food, beverages, supplements, and other drugs. For example, ingesting certain foods can increase or inhibit an amount of a drug or other chemical present in the body, as was discovered with respect to grapefruit juice and the HIV antiretroviral drug saquinavir. In particular, it was found that compounds present in grapefruit juice bind to enzymes in the liver that metabolize the drug Saquinavir, which, in turn, results in reduced excretion of the drug and increased drug effects.

While regulatory agencies require testing as to food-drug interactions, such testing requires only tests performed with respect to fed and fast conditions, without regard to the particular foods ingested by test subjects. As such, the effects of particular drug-chemical pairings are often not known until after the drug has been in use for some time.

### SUMMARY

Systems and methods are provided that can be used as tools in identifying potential drug-chemical interactions, including, for example, drug-food interactions.

A method of filtering data in a protein-protein interaction network includes mapping proteins associated with a plurality of chemicals of a first type and proteins associated with one or more chemicals of a second type. The method further includes determining proximities of proteins associated with the plurality of chemicals of the first type and proteins associated with the one or more chemicals of the second type and generating a reduced dataset of proteins within the protein-protein interaction network. The reduced dataset of proteins are proteins associated with a subset of the plurality of chemicals of the first type based on the determined proximities.

A system for filtering data in a protein-protein interaction network includes memory and a processor configured to map and store in the memory proteins associated with a plurality of chemicals of a first type and proteins associated with one or more chemicals of a second type. The processor is further configured to determine and store in memory proximities of proteins associated with the plurality of chemicals of the first type and proteins associated with the one or more chemicals of the second type and generate and store in the memory a reduced dataset of proteins within the protein-protein interaction network. The reduced dataset of proteins are proteins associated with a subset of the plurality of chemicals of the first type based on the determined proximities.

The chemicals of the first type can be drug chemicals, and the chemicals of the second type can include one or more chemical compounds found in a food, such as a polyphenol. Alternatively, the chemicals of the first type can be chemicals found in one or more foods, and the chemicals of the second type can be one or more drug chemicals. Proteins associated with the plurality of drug chemicals can be proteins that are associated with at least one of drug absorption, drug metabolism, drug distribution, and drug excretion.

The mapping of proteins can include identification of one or more drug regions within the protein-protein interaction network, or one or more disease modules within the protein-protein interaction network associated with the drug(s). The one or more drug regions can distinguish proteins associated with at least one of absorption of a drug, distribution of the drug, metabolism of the drug, and excretion of the drug from all proteins associated with the drug.

The determination of proximities can include measurement of distances, within the protein-protein interaction network, between proteins associated with the plurality of chemicals of the first type and proteins associated with the one or more chemicals of the second type. One or more proximity values can be generated for chemicals of the first and second types, or for modules comprising subsets of proteins associated with chemicals of the first and second types. For example, the proximity value can be based on a mean path distance between proteins associated with chemicals of the first and second types, such as a z-score, or the proximity value can be based on overlap of modules in the protein-protein interaction network, such as an S_{AB} score. For example, a first module can be a drug module and a second module can be a food module. Optionally, a disease module can also be included in the protein-protein interaction network. Any combination of proximity values among the modules can be determined. For example, an S_{AB} score can be provided to indicate a proximity of food to a drug, and a z-score can be used to compare the food and/or drug to Absorption, Distribution, Metabolism, and Excretion (ADME) regions or modules within the protein-protein interaction network. A ranking can be produced of chemicals associated with the subset of the plurality of chemicals of the first class based on the determined proximities.

A method of identifying a food interaction with a drug includes building a multi-layer protein-protein interaction network comprising a food-composition layer and a drug-interaction layer. The food-composition layer identifies proteins associated with one or more chemical compounds found in a food, and the drug-interaction layer comprises proteins associated with a plurality of drugs. The method further includes determining proximities of proteins in the food-composition layer and proteins in the drug-interaction layer in the protein-protein interaction network and identifying at least one food-drug interaction based on the determined proximities.

A system for identifying a food interaction with a drug includes memory and a processor configured to build and store in the memory a multi-layer protein-protein interaction network. The multi-layer network includes a food-composition layer and a drug-interaction layer. The food-composition layer identifies proteins associated with one or more chemical compounds found in a food, and the drug-interaction layer comprises proteins associated with a plurality of drugs. The processor is further configured to determine and store in the memory proximities of proteins in the food-composition layer and proteins in the drug-interaction layer in the protein-protein interaction network, and to identify at least one food-drug interaction based on the determined proximities.

The proteins associated with the plurality of drug chemicals can be proteins that are associated with at least one of drug absorption, drug metabolism, drug distribution, and drug excretion. One or more drug regions within the protein-protein interaction network can be identified, for example, drug regions that distinguish proteins associated with at least one of absorption of a drug, distribution of the drug, metabolism of the drug, and excretion of the drug from all proteins associated with the drug.

The determination of proximities can include measurement of distances, within the protein-protein interaction network, between proteins of the food-composition layer and proteins of the drug-interaction layer. Proximity values can thereby be generated. For example, a proximity value can be based on a mean path distance between proteins of the food-composition layer and the drug-interaction layer, such as a z-score, or a proximity value can be based on overlap of proteins of the food-composition layer and the drug-interaction layer, such as an S_{AB} score. A ranking of drugs of the drug-interaction layer can be produced based on the determined proximities.

While the systems and methods are generally described as providing for identification of a drug for which a food-drug interaction can occur, the systems and methods can also be used to identify a food for which a food-drug interaction can occur.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is diagram of a filter for reducing proteins of a protein-protein interaction network for a therapeutic chemical.

FIG. 2 is a diagram of a computer processor operation 100 for identifying a disease associated with a therapeutic chemical.

FIG. 3 is a schematic view of a computer network environment in which embodiments of the present invention may be deployed.

FIG. 4 is a block diagram of computer nodes or devices in the computer network of FIG. 3.

FIG. 5 is a schematic illustrating examples of proximity measurements among nodes of a protein-protein interaction network.

FIG. 6A is a schematic illustrating an example of disease nodes and drug target nodes in a protein-protein interaction network.

FIG. 6B illustrates determination of a z-score of the nodes of FIG. 6A.

FIG. 6C illustrates an interactome neighborhood and proximities of Gliclazide and Daunorubicin drug targets with respective disease gene proteins.

FIG. 7A illustrates a neighborhood of a protein-protein interaction network and proximities of various disease modules mapped within the network.

FIG. 7B illustrates an S_{AB} score of two overlapping disease modules of FIG. 7A.

FIG. 7C illustrates an S_{AB} score of two separated disease modules of FIG. 7A.

FIG. 8 illustrates a protein-protein interaction network with mapped proteins associated with drugs and foods.

FIG. 9 is a diagram illustrating example modules of a multi-layer network.

FIG. 10 illustrates a network proximity measure of proteins involved in drug metabolism and proteins that bind to a chemical, such as a food-chemical or a drug-chemical.

FIGS. 11A-11F illustrate network classifications of chemical-chemical interactions, such as food-drug interactions: FIG. 11A illustrates overlapping exposure; FIG. 11B illustrates complementary exposure; FIG. 11C illustrates indirect exposure; FIG. 11D illustrates single exposure; FIG. 11E illustrates non-exposure; and, FIG. 11F illustrates independent action.

### DETAILED DESCRIPTION

A description of example embodiments follows.

Systems and methods are presented for identifying food-drug interactions. The systems and methods presented herein can function as a filter in a protein-protein interaction network, such as the human interactome, to reduce proteins present in the network to a subset of proteins associated with a food and one or more drugs, or with a drug and one or more foods, for which interactions can occur.

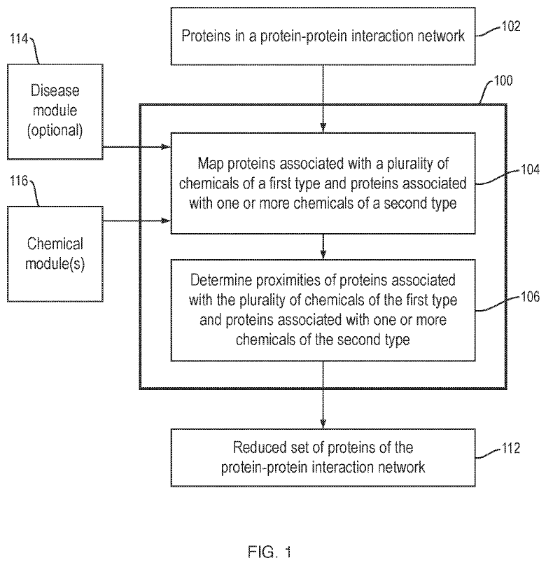

An example of a filter 100 that can be applied to a protein-protein interaction network 102 is shown in FIG. 1. From the proteins present in a protein-protein interaction network 102, the filter 100 functions to reduce the proteins present in the network to a subset of proteins that are associated with a chemical-chemical interaction, for example, a food-drug interaction. Systems and methods including filter 100 operate by mapping proteins associated with a plurality of chemicals of a first type and proteins associated with one or more chemicals of a second type (step 104). For example, the chemicals of a first type can be drug chemicals, and the proteins associated with the drug chemicals can be proteins that bind to a drug, proteins involved in drug metabolism, proteins involved in other drug pathways (e.g., absorption, distribution, and excretion), or any combination thereof. The chemicals of the second type can be chemical compounds found in food, for example, polyphenols, and the proteins associated with the food can be proteins that bind to a chemical found in the food. Alternatively, the chemicals of the first type can be food chemicals and the chemicals of the second type can be drug chemicals, or both the first and second types of chemicals can be drug chemicals.

Optionally, information regarding proteins associated with one or more diseases can be provided from a disease module 114 to identify disease clusters within the protein-protein interaction network. The disease clusters can include proteins associated with a disease to be treated by one or more of the drugs.

Information regarding proteins associated with one or more chemicals can be provided by chemical module(s) 116 to identify locations (e.g., nodes, regions) within the network comprising proteins associated with one or more chemicals. The chemical module(s) can include, for example, proteins targeted by one or more drugs, proteins involved in the metabolism of one or more drugs, proteins targeted by chemical compounds present in one or more foods, or any combination thereof. After mapping, the filter 100 determines proximities, within the network, of proteins associated with the plurality of chemicals of the first and second types (step 106). Based on the determined proximities, the proteins within the network are reduced to one or more sets 112 associated with particular chemical-chemical interaction(s), for example, particular food-drug interaction(s).

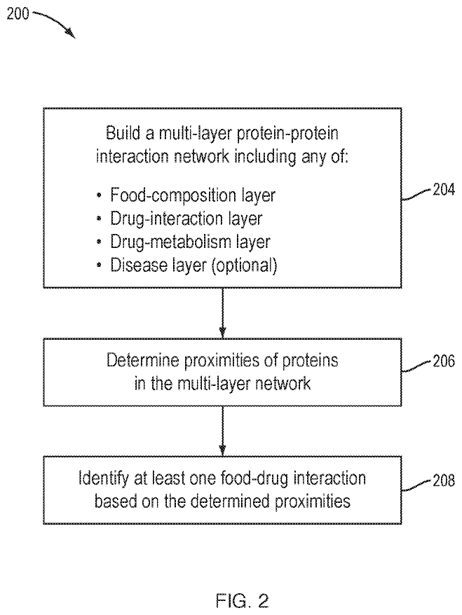

An example of a method 200 for identifying a food-drug interaction is shown in FIG. 2. The method includes building a multi-layer protein-protein interaction network (step 204). The multi-layer network can include food-composition layer(s), a drug-interaction layer(s), and, optionally, disease layer(s). Proximities of proteins within the multi-layer network can be determined (step 206), and at least one food-drug interaction can be identified based on the determined proximities (step 208).

The drug-interaction layer(s) of the network can be provided to identify proteins with which the drug interacts, either directly or indirectly. For example, a multi-layer protein-protein interaction network can include a layer that identifies proteins that bind to a drug and another layer that identifies proteins involved in metabolism of the drug. The drug interaction layer(s) can provide for identification of proteins associated with drug interactions of any type, including Absorption, Distribution, Metabolism, and Excretion (ADME) interactions. Such interactions can be interactions that positively or negatively impact therapeutic effectiveness of the drug, interactions that can result in an adverse effect, or a combination thereof. For example, interactions can be those which impact gastrointestinal absorption of a drug, binding of the drug to a plasma protein, distribution of the drug, transport of the drug through tissue, enhancement or weakening of binding of the drug to a receptor, induction or inhibition of drug metabolism, and increased or inhibited secretion of the drug.

In a particular example, for illustration purposes, it is known that chemical compounds found in grapefruit bind to CYP P450 enzymes in the liver, which are also responsible for excretion of some HIV antiretroviral drugs, such as saquinavir. A food-composition layer within a multi-layer network can provide for mapping of chemical compounds within grapefruit that bind to CYP enzymes, and a drug-interaction layer can provide for mapping of proteins associated with metabolism or excretion of saquinavir, including CYP enzymes. From measured proximities among the layers of the protein-protein interaction network, the interaction between grapefruit and saquinavir via CYP enzymes can be identified. While the food-drug interaction between grapefruit and saquinavir is known, the systems and methods described herein can be used to reveal unknown adverse food-drug interactions, and/or potential food-drug combinations that may improve therapeutic effectiveness of a drug.

Example methods and systems for providing a food-composition layer are described in US2018/0286516, the entire contents of which are incorporated herein by reference.

Example methods and systems for identifying a disease cluster within a protein network are described in W02015/084461, the entire contents of which are incorporated herein by reference.

The chemicals of the first and second type can be any chemical, including, for example, drug chemicals (e.g., pharmaceuticals, synthetic drugs), natural or food-borne chemical compounds (e.g., polyphenols, nutraceuticals, general phytochemicals present in food), and nontherapeutic chemicals, such as toxins.

The protein-protein interaction network can be, for example, the human interactome, which includes a map of protein interactions in the human cell. Other protein-protein interaction networks can be used, such as, for example, networks from STRINGDB and GeneMania databases.

Layers of a multi-layer network can include one or more layers that identify proteins involved in ADME processes of a drug, proteins targeted by a drug, chemical compounds present in a food, and proteins targeted by chemical compounds present in a food. For example, proteins involved in the metabolism of a drug can be obtained from the PharmGKB database, and such information can be used in the mapping of drug-associated proteins within the network or application of a drug-associated protein layer to the network. In further examples, proteins targeted by drugs can be obtained from the DrugBank database, proteins targeted by chemical compounds present in food can be obtained from the STITCH database, and chemical compounds present in food can be obtained from the FooDB database. Additional information relating to foods, drugs, and food-drug interactions can be retrieved from databases available at USDA/Medline, DrugBank, Drugs.com, and Nutrichem.

One or more drug modules can be included in the network. For example, modules relating to any or all of drug metabolism, efficacy, and dosage (DMED) can be included and be formed based on information obtained from, for example, the PharmGKB database. PharmGKB is a public pharmacogenomics database that reports genetic variants which affect humans' responses to medication. PharmGKB provides individual variant annotations that are divided into three categories based on the context of a discovery and its effect: phenotype variant annotations, drug variant annotations, and functional analyses variant annotations. A drug module can include information from any or all variant annotations. For example, a drug module can include information from the drug variant annotations, as these genetic variants refer to those which specifically affect drug responses. PharmGKB contains 9,717 drug variant annotations which are categorized into seven different “effects.” The effect for a given variant annotation provides another layer of resolution, as it describes more precisely how the genetic variant is affecting the drug response. The seven effects include: efficacy, dosage, metabolism/PK, toxicity, PD, other, and none.

For any or all of the seven PharmGKB effects, respective gene sets can be mapped in the protein-protein interaction network (e.g., the human interactome). A largest connected component (LCC) for each effect can be identified. Of the seven effects, three are found to be the most significant, including the efficacy effect, the dosage effect, and the metabolism/PK effect. Where statistically significant results are unable to obtained from any one of the effects, effect subgraphs can be expanded to a desired size (e.g., 100 nodes, 500 nodes, 1000 nodes), based on statistical measures.

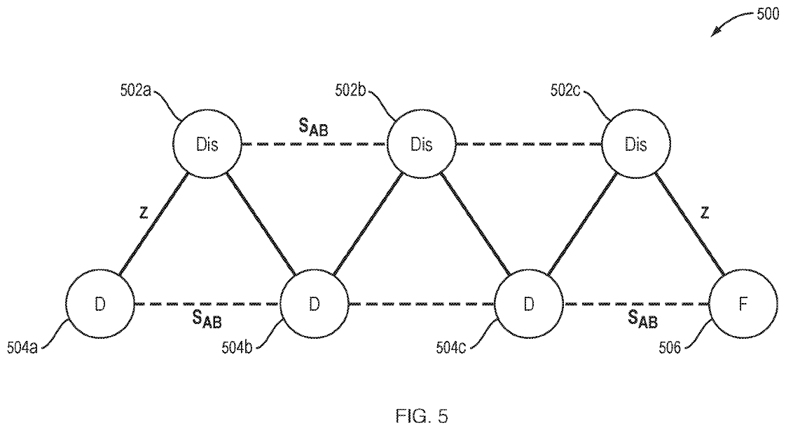

A simplified example of a protein-protein interaction network 500 is shown in FIG. 5. As illustrated, the network includes disease modules 502a-c (e.g., nodes or regions within the network that identify proteins associated with a disease), drug modules 504a-c (e.g., nodes or regions within the network that identify proteins associated with a drug), and a food module 506 (e.g., node or region within the network that identify one or more proteins associated with a food). Proximities among the proteins of the modules can be determined, including, for example, z-scores and S_{AB} scores.

One proximity measure, a z-score is determined by a shortest path (d) between proteins of two different modules. The shortest path can then be compared to a reference distribution of a random selection of proteins with the same degrees, as given by:

$$z = \frac{( d - μ )}{σ} . \qquad [ 1 ]$$

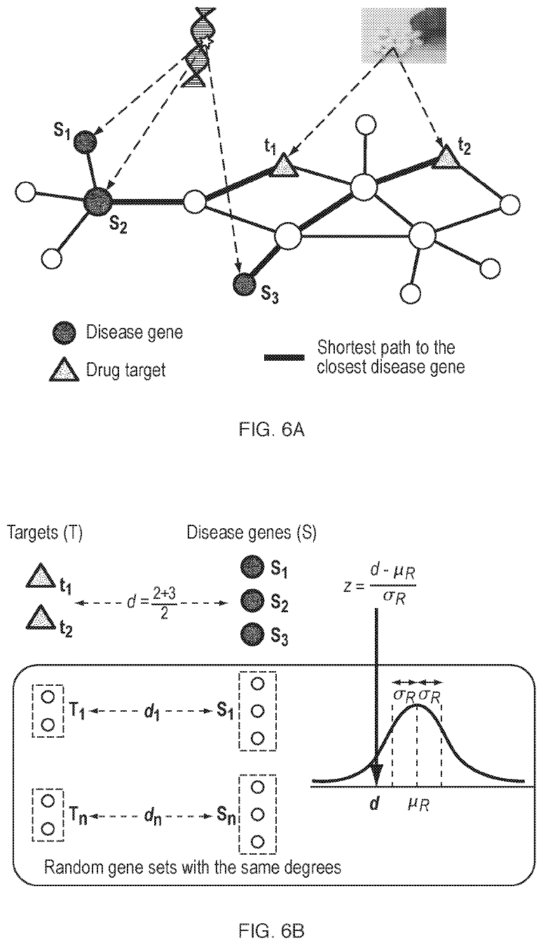

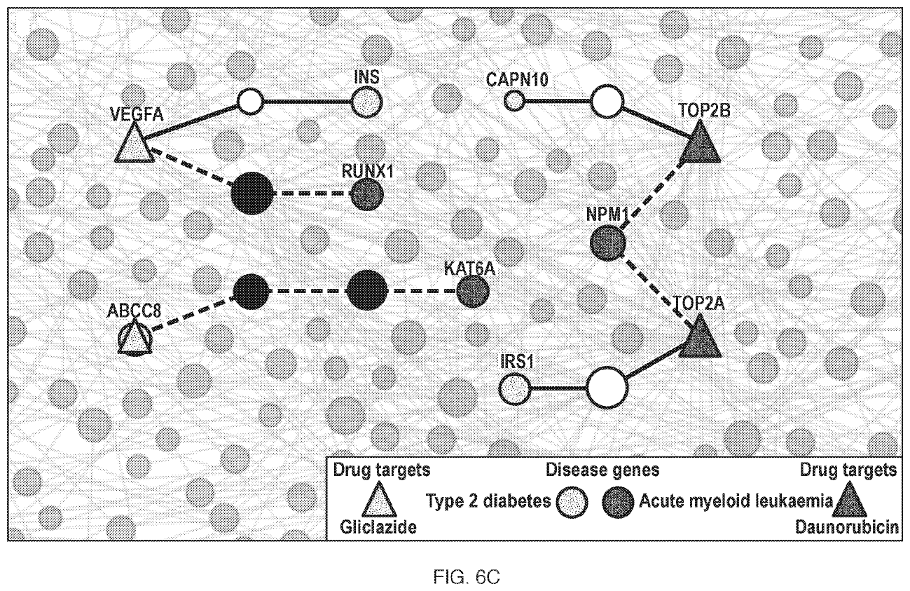

The z-score is applicable when a reference distribution is Gaussian, with μ being the mean and σ the standard deviation of the reference distribution. A low z-score indicates an interaction between two modules. For example, a low z-score between a disease module and a food module means an interaction is probable. If a drug and a food both have a low z-score to a particular disease, then a potential drug-food interaction is possible. Examples of z-scores within a network are shown in FIGS. 6A-6C, with proteins associated with a disease and proteins that are drug targets being provided for illustration.

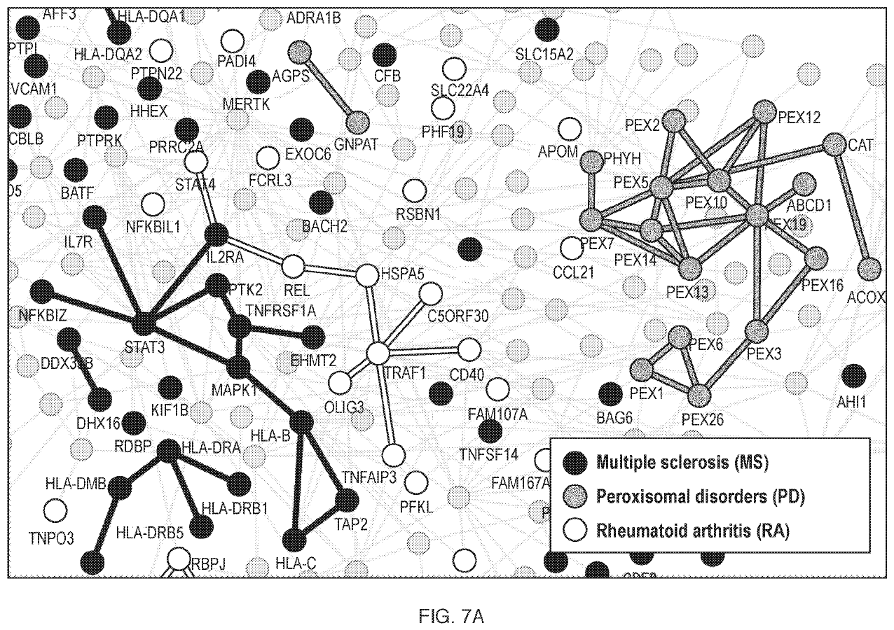

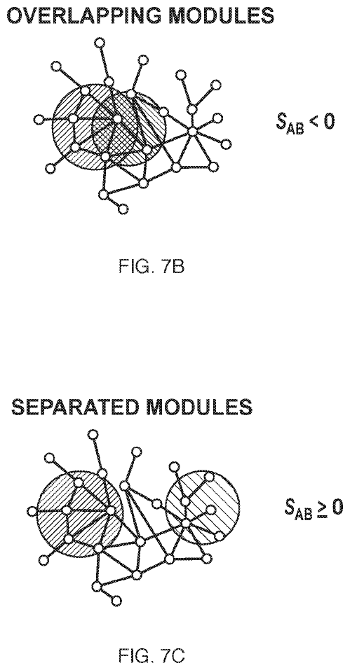

Another proximity score, s_{AB}, compares a mean shortest path between two modules and the associated targets of the two modules. For example, disease A to disease B:

$${s}_{A B} = 〈 {d}_{A B} 〉 - \frac{〈 {d}_{A A} 〉 + 〈 {d}_{BB} 〉}{2} \qquad [ 2 ]$$

where <d_{AB}> is the mean shortest path between each target of disease A to each target of disease B, and vice versa, while <d_{AA}> is the mean shortest distance between each target in disease A to each target in disease A and <d_{BB}> is between the targets of disease B. The s_{AB} can be more applicable when there is a sizable number of protein interactions to construct a module. Examples of s_{AB} scores within a network are shown in FIGS. 7A-7C, with disease modules provided for illustration. For example, as illustrated in FIGS. 7A and 7B, modules representing proteins associated with multiple sclerosis and proteins associated with rheumatoid arthritis reflect overlap within the network, and, accordingly, an s_{AB} of less than zero is obtained. In contrast, as illustrated in FIGS. 7A and 7C, modules representing proteins associated with multiple sclerosis and proteins associated with peroxisomal disorders are located in disparate regions within the network, and, accordingly, an s_{AB} of greater than zero is obtained.

Both proximity measures have predictive power, and which proximity measure is used in any particular case can be dependent on a number of factors, such as the size of the modules. Both proximity measures may also be used. Furthermore, while FIGS. 6A-7C illustrate proximity scores with examples that include disease modules, disease modules are not required. Such proximity measures can be used with respect to measurement of proximities between drug modules and food modules. Drug modules and food modules can optionally be compared to disease modules to further assess a probability of potential interactions.

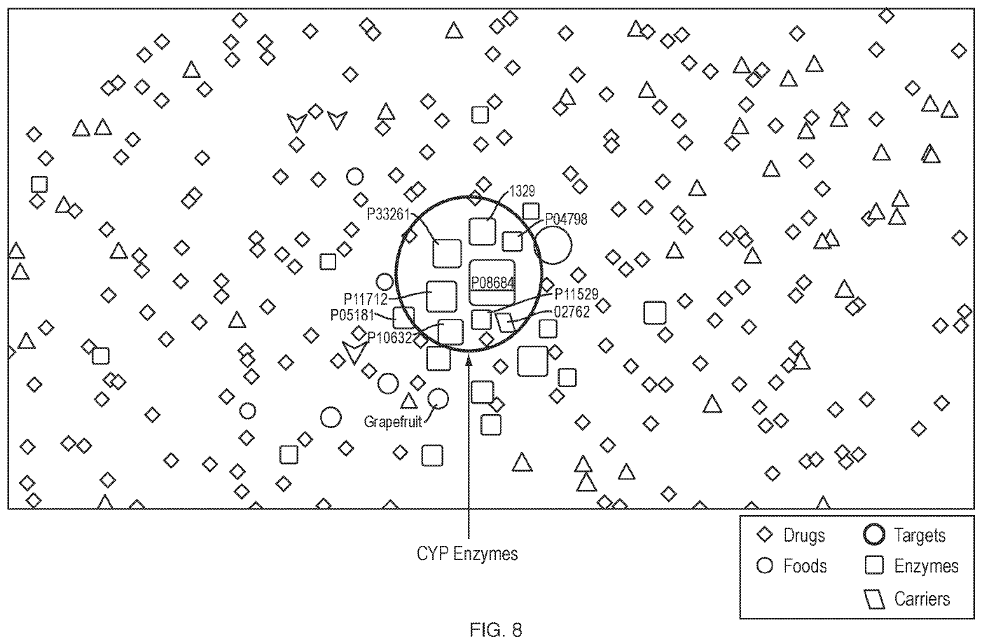

An example of a multi-layer network is shown in FIG. 8. As illustrated, the network includes a drug-layer (e.g., having nodes indicated by blue diamonds), a food layer (e.g., having nodes indicated by green circles), and drug-interaction layer (e.g., having nodes indicated by orange squares). As illustrated, a grapefruit node 606 is in close proximity with a module 608 encompassing CYP enzymes. Identification of drugs potentially impacted by grapefruit can be derived from measured proximities to the module 608.

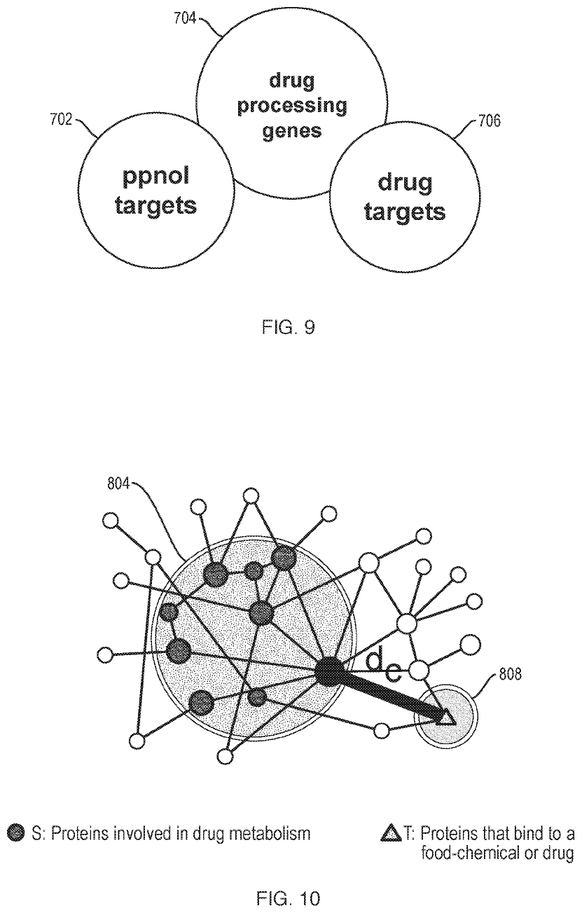

A diagram illustrating example modules/layers of a multi-layer network is shown in FIG. 9. In this example, proteins targeted by each chemical compound found in a food are represented by a polyphenol target layer 702, proteins involved in drug metabolism are represented by a drug processing layer 704, and proteins targeted by each drug are represented in a drug target layer 706. To identify potential food-drug interactions, proximities of the proteins of each module within the human interactome are obtained. The determination of proximities within the network can include determination of one or more proximity values among the several proteins (e.g., as represented by individual protein nodes and/or by modules/regions within the network that comprise several related proteins).

For example, a network proximity measurement (e.g., z-closest score) can be obtained between proteins targeted by each chemical compound in food and the proteins involved in drug metabolism, as well as between proteins targeted by each drug and the proteins involved in drug metabolism. A network overlap measurement (e.g., s_{AB} score) can be obtained between proteins targeted by each chemical compound in food and the proteins targeted by each drug. With both network proximity and network overlap measurements, chemical-drug pairs can be classified to identify predicted food-drug interactions.

An example of a network proximity measurement (d_{c}) between a drug metabolism module 804 and a binding protein 808 (e.g., a protein that binds to a food-chemical and/or to a drug) is shown in FIG. 10. The network proximity measurement can provide for the closest z-score (i.e., z-closest score) of a protein within the drug metabolism module 804 and the binding protein 808. The z-closest score (dᵣ) can be provided by:

$${d}_{c} ( S , T ) = \frac{1}{‖ T ‖} {\sum }_{t ∈ T} {min}_{s ∈ S} d ( s , t ) \qquad [ 3 ]$$

where S represents the set of proteins involved in drug-metabolism and T represents the set of binding proteins.

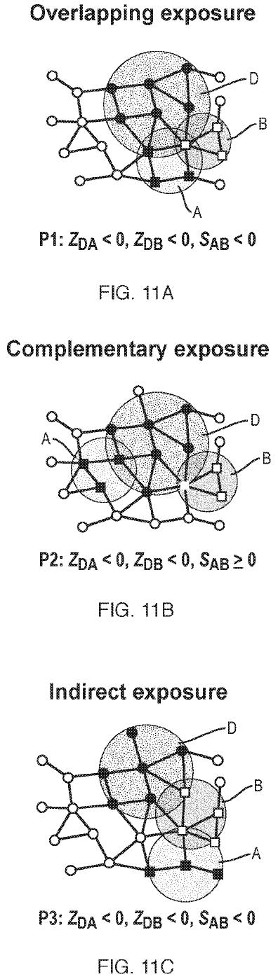

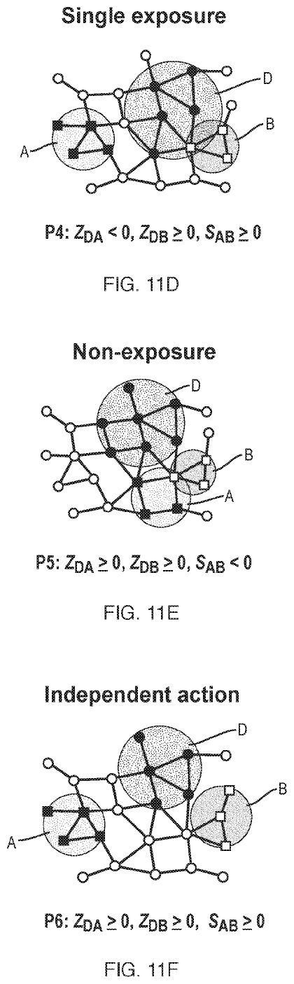

The network proximity measurement(s) can be combined with network overlap measurement(s) to classify chemical-drug pairs, as shown in FIGS. 11A-F. As illustrated, module D includes proteins involved in drug metabolism, module A includes proteins that bind to a food chemical, and module B includes proteins that bind to a drug. Where overlapping exposure (FIG. 11A) and complementary exposure (FIG. 11B) are determined between a drug and at least one chemical associated with a food, a potential food-chemical drug interaction is identified. A determination of indirect exposure (FIG. 11C), single exposure (FIG. 11D), non-exposure (FIG. 11E), and independent action (FIG. 11F) can provide an indication that a food-chemical drug interaction is unlikely to occur.

The systems and methods described can advantageously provide for identification or estimation of potential interactions between chemicals of two types, such as, for example, food-drug interactions. Drug interactions can include reactions that occur directly or indirectly with other chemicals (e.g., chemicals found in foods) that can affect how a drug works or that can result in a side effect. Drug interactions include drug-drug interactions, drug-food interactions, drug-supplement interactions, and drug-condition interactions. The drug interaction can be an interaction that increases or decreases an action of the drug.

Currently in drug development, the study of drug-food interactions is dominated around a group of proteins, termed the CYP enzymes, which are responsible for metabolizing many of the currently available drugs. Recently, attention has been given to a group of transporter proteins. Regulatory agencies only require examination of a drug under a fed state and a fast state, and pharmaceutical companies typically only examine drugs against a pallet of selected proteins that largely comprises the CYP enzymes and select transporter proteins. The methods and systems described herein can provide predictions as to which foods certain drugs may interact with, thereby providing for more precise study of possible drug-food interactions and further expanding knowledge of food-drug interactions beyond the limited proteins typically tested.

The methods and systems described herein can also provide for identification of food-drug and/or drug-drug interactions during a drug development process, or can be used by doctors and nutritional specialists to avoid potentially negative food-drug interactions that can reduce the therapeutic effectiveness of a drug being taken by a patient. Alternatively, or in addition, the systems and methods disclosed herein can be used to identify potentially positive food-drug interactions that can improve the therapeutic effectiveness of a drug. In some instances, the failure of a drug to be taken to market may be due to adverse effects that occur due to an unknown food-drug interaction, and the methods and systems described herein may be used to identify such interactions.

Moreover, such methods and systems can guide studies of drug-food interactions during drug development, before experimentation. Instead of a blind study focusing on only CYP enzymes, drug studies can target proteins in which a drug-food interaction is most likely to occur, such as by targeting the proteins provided in a reduced set by filter 100.

The methods and systems described can also provide for identification of drug-food interactions which would have been unknown under current development processes. Furthermore, such methods and systems can be used to provide for therapeutic combinations in which a concentration or a dosage of a drug is reduced while maintaining a similar therapeutic effect when taken with a particular food.

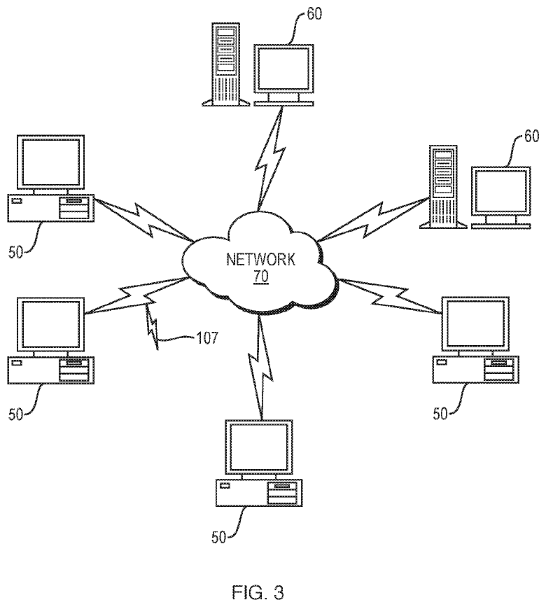

FIG.3 illustrates a computer network or similar digital processing environment in which the systems and methods described may be implemented. Client computer(s)/devices/exercise apparatuses 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), a worldwide collection of computers, cloud computing servers or service, Local area or Wide area networks, and gateways that currently use respective protocols (TCP/IP, Bluetooth, etc.) to communicate with one another. Other electronic device/computer network architectures are suitable.

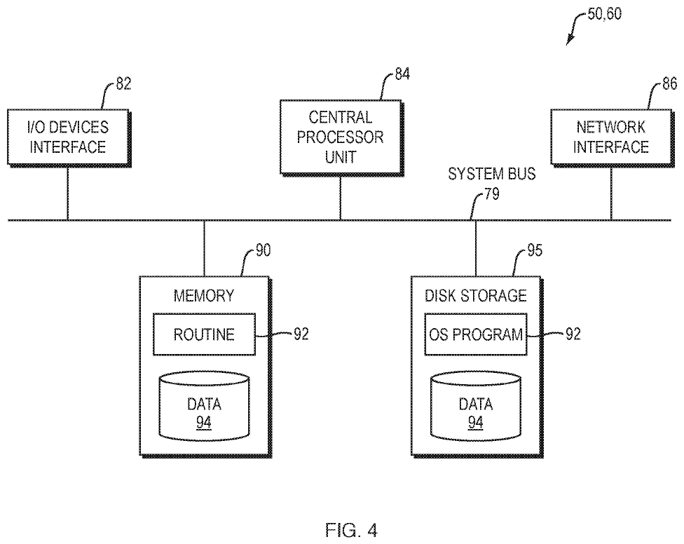

FIG. 4 is a diagram of the internal structure of a computer (e.g., client processor/device 50 or server computers 60) in the computer network of FIG. 3. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 3). Memory 90 provides volatile storage for computer software instructions 92 and data 94 used to implement embodiments of the present invention (e.g., processor routines and code for creating a directed acyclic graph (DAG) as a function of computed alignment indices and aligning sequence reads against the DAG being developed, as described herein). Disk storage 95 provides nonvolatile storage for computer software instructions 92 and data 94 used to implement an embodiment of the present invention. Central processor unit 84 is also attached to system bus 79 and provides for the execution of computer instructions.

In particular, embodiments of the present invention execute processor routines for the filter 100 and method 200 of FIGS. 1 and 2, respectively. In one embodiment, the processor routines 92 and data 94 are a computer program product (generally referenced 92), including a non-transitory computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, other mediums and the like.

In other embodiments, the computer program product 92 provides Software as a Service (SaaS) or similar operating platform.

Alternative embodiments can include or employ clusters of computers, parallel processors, or other forms of parallel processing, effectively leading to improved performance, for example, of generating a computational model. Given the foregoing description, one of ordinary skill in the art understands that different portions of processor routine 100 and different iterations operating on respective sequence reads may be executed in parallel on such computer clusters or parallel processors.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of filtering data in a protein-protein interaction network, comprising:
mapping proteins associated with a plurality of chemicals of a first type and proteins associated with one or more chemicals of a second type;
determining proximities of proteins associated with the plurality of chemicals of the first type and proteins associated with the one or more chemicals of the second type; and
generating a reduced dataset of proteins within the protein-protein interaction network, the reduced dataset of proteins being proteins associated with a subset of the plurality of chemicals of the first type based on the determined proximities.

2. The method of claim 1, wherein the chemicals of the first type comprise drug chemicals.

3. The method of claim 2, wherein the proteins associated with the plurality of drug chemicals are proteins that are associated with at least one of drug absorption, drug metabolism, drug distribution, and drug excretion.

4. The method of claim 1, wherein the one or more chemicals of the second type comprise at least one chemical compound found in a food.

5. The method of claim 4, wherein the at least one chemical compound found in a food is a polyphenol.

6. The method of claim 1, wherein mapping proteins associated with the plurality of chemicals of the first type includes identifying one or more drug regions within the protein-protein interaction network.

7. The method of claim 6, wherein the one or more drug regions distinguishes proteins associated with at least one of absorption of a drug, distribution of the drug, metabolism of the drug, and excretion of the drug from all proteins associated with the drug.

8. The method of claim 1, wherein determining proximities includes measuring distances, within the protein-protein interaction network, between proteins associated with the plurality of chemicals of the first type and proteins associated with the one or more chemicals of the second type.

9. The method of claim 8, further comprising generating a proximity value for a chemical of the first type and a chemical of the second type.

10. The method of claim 9, wherein the proximity value is based on a mean path distance between proteins associated with chemicals of the first and second types.

11. The method of claim 10, wherein the proximity value is a z-score.

12. The method of claim 8, further comprising generating a proximity value for a first module and a second module, wherein the first module comprises a subset of proteins associated with one of the plurality of chemicals of the first type and the second module comprises a subset of proteins associated with one or more chemicals of the second type.

13. The method of claim 12, wherein the first module is a drug module and the second module is a food module.

14. The method of claim 12, wherein the proximity value is based on overlap of the first and second modules in the protein-protein interaction network.

15. The method of claim 14, wherein the proximity value is an SAB score.

16. The method of claim 1, further comprising ranking chemicals associated with the subset of the plurality of chemicals of the first class based on the determined proximities.

17. A method of identifying a food interaction with a drug, comprising:
building a multi-layer protein-protein interaction network comprising a food-composition layer and a drug-interaction layer, the food-composition layer identifying proteins associated with one or more chemical compounds found in a food, the drug-interaction layer comprising proteins associated with a plurality of drugs;
determining proximities of proteins in the food-composition layer and proteins in the drug-interaction layer in the protein-protein interaction network; and
identifying at least one food-drug interaction based on the determined proximities.

18.-27. (Canceled)

28. A system for filtering data in a protein-protein interaction network, comprising:
memory; and
a processor configured to:
map and store in the memory proteins associated with a plurality of chemicals of a first type and proteins associated with one or more chemicals of a second type;
determine and store in the memory proximities of proteins associated with the plurality of chemicals of the first type and proteins associated with the one or more chemicals of the second type; and
generate and store in the memory a reduced dataset of proteins within the protein-protein interaction network, the reduced dataset of proteins being proteins associated with a subset of the plurality of chemicals of the first type based on the determined proximities.

29. (canceled)

30. (canceled)

31. (canceled)

32. (canceled)

33. The system of claim 28, wherein the processor is further configured to identify one or more drug regions within the protein-protein interaction network.

34. (canceled)

35. The system of claim 28, wherein the processor is further configured to measure distances, within the protein-protein interaction network, between proteins associated with the plurality of chemicals of the first type and proteins associated with the one or more chemicals of the second type for determination of proximities.

36.-53. (Canceled)
